(19) United States
(12) Patent Application Publication
(10) Pub. No.: US 2003/0134781 A1
(43) Pub. Date: Jul. 17, 2003

(54) Methods for the treatment of hyperpigmentation of skin

(75) Inventors: Robin Carmichael, Redmond, WA (US); Leonard M. Patt, Seattle, WA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: ProCyte Corporation, Redmond, WA

(21) Appl. No.: 10/264,392

(22) Filed: Oct. 4, 2002

Related U.S. Application Data
(60) Provisional application No. 60/364,657, filed on Mar. 14, 2002.
(60) Provisional application No. 60/327,640, filed on Oct. 5, 2001.

(51) Int. Cl. A61K 38/16; A61K 38/06

(52) U.S. Cl. 514/6

(57) ABSTRACT

Methods for treating hyperpigmentation of skin employ topical application of compositions to skin in need thereof, where the compositions comprise at least one peptide copper complex, or at least one peptide copper complex in combination with retinol, at least one retinol derivative, or a mixture thereof. Also disclosed are methods that use such compositions further comprising active agents selected from active drug substances, emollients, sunscreen agents, skin-lightening agents, skin protectants, skin conditioning agents, and humectants.

### CROSS-REFERENCE TO RELATED APPLICATION

This application claims the benefit of U.S. Provisional Patent Application No. 60/364,657 filed Mar. 14, 2002 and U.S. Provisional Patent Application No. 60/327,640 filed Oct. 5, 2001, where the two provisional applications are incorporated herein by reference in their entireties.

### BACKGROUND OF THE INVENTION

1. Field of the Invention

The present invention generally relates to the treatment of dermatological conditions, more specifically, of those conditions related to hyperpigmentation, by means of the topical application of compositions, effective therefor.

2. Description of the Related Art

Melanin is a dark pigment found in the skin of humans that is responsible for the darkening of the skin. Melanin is produced by specialized cells in the skin called malanocytes through a complicated series of chemical and enzymatic reactions, mainly involving the copper containing enzyme tyrosinase. The melanin pigments are packaged in granules called melanosomes. Melanosomes are transferred to the outer layer of the skin where they are responsible for the darkening of the skin, the degree of darkening being associated with skin type, sun exposure, and/or certain dermatological conditions.

There are several dermatological conditions associated with unwanted or excessive production of melanin. Conditions associated with overproduction of melanin are termed hyperpigmentation. For example, melanosis or melasma is a condition characterized by the development of sharply demarcated blotchy, brown spots usually in a symmetric distribution over the cheeks, forehead, and sometimes on the upper lip and neck. This condition frequently occurs during pregnancy (melasma gravidarum or “mask of pregnancy”), and at menopause. Also, this condition is frequently found among those taking oral contraceptives, and occasionally found among nonpregnant women who are not taking oral contraceptives, and sometimes among men. A pattern of facial hyperpigmentation, similar to that described above, may be associated with a chronic liver disease called chloasma.

One common condition associated with aging skin is the development of dark spots sometimes referred to as “liver spots.” Other forms of hyperpigmentation can be caused by UV irradiation, or result from a genetic predisposition for the condition, or may come about during the course of wound healing.

With regard to treating the above conditions, Applicants note that copper, when complexed with a biologically acceptable carrier molecule, is known to stimulate the accumulation of collagen and elastin, increase the rate of wound healing, and increase the amount of collagen in skin. For example, peptide copper complexes that are useful for wound healing and skin health are disclosed in U.S. Pat. Nos. 4,760,051; 4,665,054; 4,877,770; 5,135,913 and 5,348,943, as well as in U.S. Patent Application No. 60/327,371.

Also, the topical use of retinol (vitamin A) and retinol derivatives in the treatment of aged or photodamaged skin has been reported. For example, the retinol derivative, retinoic acid (present in Retin-A and Renova, Ortho Pharmaceuticals, Skillman, N.J.), has been shown to reduce the signs of photoaging (see J. Invest. Dermatology 104(4): 518-522, 1995). Retinoic acid compositions, useful in skin treatment and cosmetic preparations, have been disclosed, for example, in U.S. Pat. Nos. 5,955,109; 5,719,195 and 4,126,693.

Various compositions used for skin care applications comprising retinol, retinol derivatives, or mixtures thereof, in combination with other constituents, have also been described. For example, compositions containing fatty acid amides in addition to retinol or retinyl ester are described in U.S. Pat. No. 5,811,110. As another example, compositions containing geranyl geraniol in addition to retinol or retinyl esters are described in U.S. Pat. No. 5,756,109. As yet another example, U.S. Pat. No. 5,738,858 describes compositions containing fatty hydroxyethyl imidazoline surfactants, in addition to retinol or retinol.

Additionally, a number of compounds and plant extracts are reported to have activity against hyperpigmentation, including ascorbic acid and derivatives thereof, kojic acid and compounds related thereto, licorice (glycyrrhiza) extract, and bearberry extract. While these chemical compounds and extracts are active in the reversal and prevention of hyperpigmentation, they can be irritating to the skin with prolonged use. The only drug approved for the treatment of hyperpigmentation is hydroquinone.

Accordingly, there remains a need in the art for improved, more effective methods for treating dermatological conditions related to hyperpigmentation by, for example, topically applying compositions, having a desired degree of effectivity, to areas of skin in need thereof. The present invention fulfills this need and provides further related advantages.

### BRIEF SUMMARY OF THE INVENTION

In brief, the present invention is directed to treating dermatological conditions related to hyperpigmentation of skin by means of the topically applying thereto compositions comprising at least one peptide copper complex. It has been surprisingly found that such compositions can be used topically to substantially diminish signs of hyperpigmentation found in, for example, aging skin.

In one embodiment, the present invention is directed to a method for treating hyperpigmentation of skin, by topically applying to areas of skin in need thereof an effective amount of a composition comprising a peptide copper complex. In another embodiment, the composition further comprises retinol, a retinol derivative, or a mixture thereof. Topical application of an effective amount of the disclosed compositions to areas of skin in need of such treatment, results in significant reduction of the hyperpigmentation found on the areas contacted.

Additional embodiments of the present invention are directed to methods for such treatment where the peptide copper complex is encapsulated in a liposome or microsponge, or where the peptide copper complex is formulated in an instrument adapted to deliver the complex via iontophoresis. In yet further related embodiments directed to methods for such treatment, the compositions used also provide cosmetic preparations for skin, where the compositions further comprise an inert and physiologically-acceptable carrier or diluent, a skin-lightening agent, a sunscreen agent, a skin conditioning agent, a skin protectant, an emollient, a humectant, or a mixture thereof. In another related embodiment, the composition used to treat hyperpigmentation further comprises at least one active drug substance to, thereby, also provide a pharmaceutical preparation for skin.

The present invention is also directed to methods for treating hyperpigmentation where the compositions used are formulated as emulsions and, accordingly, further comprise emulsifiers and surfactants. In certain specific embodiments, the compositions further comprise thickening or viscosity increasing agents, and in other specific embodiments, the compositions further comprise suitable excipients, and are in the form of a solution, cream, gel, fluid cream or milk, lotion, or oil. These and other aspects of this invention will be evident upon reference to the following detailed description of the invention.

### DETAILED DESCRIPTION OF THE INVENTION

As noted above, in one embodiment, disclosed is a method for treating hyperpigmentation of skin by topically applying to areas of skin in need thereof, an effective amount of a composition comprising at least one peptide copper complex. As used herein the word “treat,” “treating” or “treatment” refers to using the compositions of the present invention either prophylactically to prevent hyperpigmentation, or to ameliorate an existing condition characterized by hyperpigmentation. In another embodiment, the composition used further comprises retinol, at least one retinol derivative, or a mixture thereof

The compositions used for the above embodiments may be in any form suitable for topical application, including: a cream, a lotion, a gel and a solution. Some examples of compositions formulated as cosmetic preparations, useful for cleansing and protecting, in addition to treating, skin are: creams for the face, hands, feet, or the entire body (i.e., day creams, night creams, make-up removal creams, and foundation creams); make-up removal formulations; protective or skin care body milks; skin care lotions, gels, or foams (such as cleansing or disinfecting lotions); bath compositions; deodorant compositions; and aftershave and preshave gels or lotions.

As used herein, the term “peptide copper complex” refers to a coordination compound comprising a peptide molecule and a copper ion non-covalently complexed therewith. The peptide molecule serves as the complexing agent by donating electrons to the copper ion to yield the non-covalent complex. The peptide molecule is a chain of two or more amino acid units covalently bonded together via amide linkages (for example, —CONH—), the formation of such linkages being accompanied by the elimination of water. The amino acid units are from amino acids that are naturally occurring or otherwise. Also, at least one amide linkage nitrogen atom may have covalently bonded thereto either a hydrogen atom or another moiety.

Generally, an amino acid consists of an amino group, a carboxyl group, a hydrogen atom, and an amino acid side-chain moiety—all bonded, in the case of an alpha-amino acid, to a single carbon atom that is referred to as an alpha-carbon. The amino acid units of the peptide copper complexes used for the methods of the present invention may be provided by amino acids other than alpha-amino acids. For example, the amino acids may be beta- or gamma-amino acids, such as those shown below.

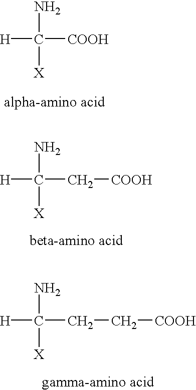

where X is the amino acid side-chain moiety.

Naturally occurring amino acids, that is, amino acids from which the amino acid units of naturally occurring proteins are derived, and their respective naturally occurring, amino acid side chain moieties, are shown below in Table 1. These naturally occurring amino acids are all in the L configuration, referring to the optical orientation of the alpha carbon or other carbon atom bearing the amino acid side chain. A peptide molecule may also comprise amino acids that are in the D optical configuration.

TABLE 1
NATURALLY OCCURRING AMINO ACID SIDE-CHAIN
MOIETIES

| Amino Acid Side Chain Moiety | Amino Acid |
|---|---|
| —H | Glycine |
| —CH₃ | Alanine |
| —CH(CH₃)₂ | Valine |
| —CH₂CH(CH₃)₂ | Leucine |
| —CH(CH₃)CH₂CH₃ | Isoleucine |
| —(CH₂)₄NH₃⁺ | Lysine |
| —(CH₂)₃NHC(NH₂)NH₂⁺ | Arginine |
| 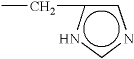 | Histidine |
| —CH₂COO— | Aspartic Acid |
| —CH₂CH₂COO— | Glutamic Acid |
| —CH₂CONH₂ | Asparagine |
| —CH₂CH₂CONH₂ | Glutamine |
| 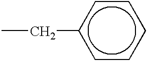 | Phenylalanine |
| 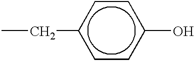 | Tyrosine |
| 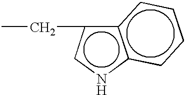 | Tryptophan |
| —CH₂SH | Cysteine |
| —CH₂CH₂SCH₃ | Methionine |
| —CH₂OH | Serine |
| —CH(OH)CH₃ | Threonine |

One example of a copper peptide complex is alanyl-histidyl-lysine:copper(II). Copper(II), as is well understood by the skilled artisan, designates a copper ion having a valence of 2 (e.g., Cu⁺²). Additional examples of the peptide copper complexes, encompassed in embodiments of the present invention, include, but are not limited to, those described in U.S. Pat. Nos. 4,665,054; 4,760,051; 4,767,753; 4,877,770; 5,023,237; 5,059,588; 5,120,831; 5,135,913; 5,145,838; 5,177,061; 5,214,032; 5,348,943; 5,538,945 and 5,550,183, incorporated herein by reference in their entireties.

Copper is known to have many beneficial biological applications and effecting cosmetic improvements by, or example, stimulating a variety of processes related to skin, such as collagen, elastin and glycosaminoglycan production (see, e.g., Maquart, F. X., Pickart, L., Laurent, M., Gillery, P., Monboisse, J. C., Borel, J. P., “Stimulation of Collagen Synthesis in Fibroblast Cultures by the Tripeptide-Copper Complex Glycyl-L-Histidyl-L-Lysine-Copper(2+),” FEBS Lett. 238(2): 343-346, 1988; Wegrowski, Y., Maquart, F. X. and Borel, J. P., “Stimulation of Sulfated Glycosaminoglycan Synthesis by the Tripeptide-Copper Complex Glycyl-L-Histidyl-L-Lysine-Copper(2+),” Life Sciences 51: 1049-1056, 1992; Maguart, F. X., Bellon, G., Chaqour, B., Wegrowski, J., Patt L. M., Trachy, R. E., Monboisse, J. C., Chastang, F., Birembaut, P., Gillery, P. and Borel, J. P., “In Vivo Stimulation of Connective Tissue Accumulation by the Tripeptide-Copper Complex Glycyl-L-Histidyl-L-Lysine-Copper(2+) in Rat Experimental Wounds,” J. Clin. Invest. 92: 2368-2376, 1993). The above-cited references are incorporated herein by reference in their entireties.

Copper salts alone are ineffective, or even inhibitory, for such applications. The copper must be delivered in a biologically acceptable form. As an example, when copper is complexed with a biologically acceptable carrier molecule, such as a peptide, it may then be effectively delivered to cells.

In certain specific embodiments of the method of the present invention, the at least one peptide copper complex used therefor is alanyl-histidyl-lysine:copper(II) (“AHK-Cu”), valyl-histidyl-lysine:copper(II) (“VHK-Cu”), or glycyl-histidyl-lysine:copper(II) (GHK-Cu”). As is well understood in the art, copper(II) designates a copper ion having a valence of 2 (e.g., Cu⁺²). Further, such peptides may be in either the L or D form. In a related, more specific embodiment, they are all in the L form.

Further, the expression “peptide copper complex,” as used herein, encompasses peptide copper complex derivatives. The expression “peptide copper complex derivative,” as used herein, refers to a peptide copper complex where the peptide molecule thereof has: 1) at least one amino acid side chain moiety that is a modification and/or variation of a naturally occurring, amino acid side-chain moiety; and/or 2) at least one of the hydrogens, bonded to an amide linkage nitrogen atom, substituted with a different moiety; and/or 3) the carboxyl group of the carboxyl terminal residue esterified or otherwise modified; and/or 4) at least one hydrogen, bonded to the nitrogen atom of the amino-terminal residue, substituted with a different moiety.

The amino acid side-chain moieties of the peptide copper complex derivatives may include alkyl, aryl, arylalkyl, alkoxy, or aryloxy moieties. As used herein, “alkyl” means a straight chain or branched, cyclic or noncyclic, substituted or unsubstituted, saturated or unsaturated aliphatic hydrocarbon containing from 1 to 18 carbon atoms. Representative saturated straight chain alkyls include methyl, ethyl, n-propyl and the like; while saturated branched alkyls include isopropyl, sec-butyl, isobutyl, tert-butyl, isopentyl, and the like. Representative, saturated cyclic alkyls include cyclopropyl, cyclobutyl, cyclopentyl, —CH₂cyclohexyl, and the like; while unsaturated cyclic alkyls include cyclopentenyl, cyclohexenyl, and the like. Unsaturated alkyls contain at least one double or triple bond between adjacent carbon atoms (referred to as an “alkenyl” or “alkynyl,” respectively). Representative alkenyls include ethylenyl, 1-butenyl, isobutylenyl, 2-methyl-2-butenyl, and the like; while representative alkynyls include acetylenyl, 2-butynyl, 3-methyl-1-butynyl, and the like.

Also, as used herein, “aryl” means an aromatic carbocyclic moiety such as phenyl or naphthyl, and may be substituted or unsubstituted. “Arylalkyl,” as used herein, means an alkyl having at least one alkyl hydrogen atom replaced with a substituted or unsubstituted aryl moiety, such as benzyl (i.e., —CH₂phenyl, —(CH₂)₂phenyl, —(CH₂)₃phenyl, —CH(phenyl)₂, and the like). As some examples, the amino acid side-chain moieties of alanine, valine, leucine, isoleucine and phenylalanine may generally be classified as alkyl, aryl or arylalkyl moieties.

“Alkoxy” and “aryloxy,” as used herein, refer, respectively, to alky and aryl moieties, as defined above, but each further comprising an oxygen atom used to link the moiety to the amino acid.

Additionally, the peptide copper complex derivative may, for example, be N-alkylated at one or more peptide bonds; and/or its carboxyl terminus may be esterified, for example, with a methyl, ethyl, or benzyl group, or may be reduced to a hydroxy or aldehyde. Additionally, the peptide copper complex derivative may, for example, be N-alkylated, N-acylated or N-sulfonylated at the amino terminus with, for example, methyl, benzyl, acetyl, benzoyl, methanesulfonyl, or fluorenyloxycarbonyl moieties.

Examples of the peptide copper complex derivatives, encompassed in embodiments of the present invention, include, but are not limited to, those disclosed and described in the above-cited U.S. Patents that are directed to peptide copper complexes, as well as those disclosed and described in the published PCT application having the international publication number WO 94/03482, incorporated herein by reference in its entirety.

In one specific embodiment, the method of the present invention uses a composition comprising a peptide copper complex derivative that is a derivative of GHK-Cu having the general formula:

[glycyl-histidyl-lysine-R]:copper(II)

where R is an alkyl moiety containing from 1 to 18 carbon atoms, an aryl moiety containing from 6 to 12 carbon atoms, an arylalkyl moiety, an alkoxy moiety containing from 1 to 12 carbon atoms, or an aryloxy moiety containing from 6 to 12 carbon atoms. This derivative of GHK-Cu is further described in the above-cited U.S. patents that are directed to peptide copper complexes.

The above compositions may be prepared from aqueous solutions of peptide copper complexes. Such solutions are prepared by methods that are well known to those skilled in the art. For example, an amount of dried peptide copper complex suitable for a desired concentration is readily dissolved in water with mixing and gentle heating. An alternative method is to prepare a solution of the desired peptide, followed by the addition of a copper salt in the desired molar ratio to yield the desired solution of the peptide copper complex. Examples of copper salts that may be used are cupric chloride and cupric acetate. When aqueous solutions of peptide copper complexes are prepared, the solutions are neutralized, typically with NaOH.

In particular embodiments directed to disclosed methods, as generally described above, for treating hyperpigmentation of the skin, the compositions used therefor comprise at least one peptide copper complex at a concentration, by weight of the composition, ranging from about 0.01% to about 5%, from about 0.025% to about 1%, and from about 0.05% to about 0.5%, respectively. Also, the molar ratio of peptide to copper in the peptide copper complex ranges from about 1:1 to about 3:1 in some embodiments, and from about 1:1 to about 2:1 in other embodiments.

In yet another related embodiment, the composition used comprises at least one peptide copper complex that is formulated in an instrument allowing the delivery of the peptide copper complex via iontophoresis to the area of skin in need of treatment.

As noted above, certain embodiments of the methods of the present invention use compositions that comprise retinol, a retinol derivative, or a mixture thereof, in addition to a peptide copper complex. Retinol is also known as vitamin A and has the formula 3,7-dimethyl-9-(2, 6, 6-trimethyl-1-cyclohexen-1-yl)-2,4,6,8-nonatetraen-1-ol. Other terms that are used for retinol are axerophthol and vitamin A alcohol. In certain specific embodiments of the present invention that use compositions comprising retinol, the isomeric forms of the retinol used are: all-trans-retinol; 1,3-cis-retinol; 3,4-didehydro-retinol; and 9-cis-retinol, respectively. In other specific embodiments of the present invention that use compositions comprising a retinol derivative, the latter is an ester of retinol selected from C₁-C₃₀ esters of retinol; C₂-C₂₀ esters of retinol; and C₂, C₃, and C₁₆ esters of retinol, respectively. More specifically, the ester of retinol may be retinyl palmitate, retinyl acetate and retinyl propionate. Other retinol derivatives that may be used are retinoic acid or retinyl aldehyde. The concentration of the retinol, retinol derivative, or mixture thereof, ranges from about 0.001% to about 10% in some embodiments; from about 0.01% to about 1% in other embodiments; and from about 0.01% to about 0.5% in yet other embodiments, by weight of the composition.

In further particular embodiments of the methods of the present invention, the compositions used therefor may comprise at least one active agent in addition to a peptide copper complex, and retinol, a retinol derivative, or a mixture thereof. In one such embodiment, the composition is formulated as a pharmaceutical preparation and comprises at least one active drug substance. In another such embodiment, the composition further comprises at least one active agent for rendering the composition suitable as a cosmetic preparation. Active agents, as defined herein, are compounds that provide benefits to the skin and/or provide desirable properties to a composition formulated as a cosmetic preparation. Some examples of active agents, other than drug substances, are sunscreen agents, skin-lightening agents, tanning agents, skin conditioning agents, skin protectants, emollients and humectants.

The sunscreen agents included in compositions used for certain embodiments of the present invention are active ingredients that absorb, reflect, or scatter radiation in the UV range at wavelengths from 290 to 400 nanometers. Specific examples include benzophenone-3 (oxybenzone), benzophenone-4 (sulisobenzone), benzophenone-8 (dioxybenzone), butyl methoxydibenzoylmethane (Avobenzone), DEA-methoxycinnamate (diethanolamine methoxycinnamate), ethyl dihydroxypropyl PABA (ethyl 4-[bis(hydroxypropyl)] aminobenzoate), ethylhexyl dimethyl PABA (Padimate O), ethylhexyl methoxycinnamate (octyl methoxycinnamate), ethylhexyl salicylate (octyl salicylate), homosalate, menthyl anthranilate (Meradimate), octocrylene, PABA (aminobenzoic acid), phenylbenzimidazole sulfonic acid (Ensulizole), TEA-salicylate (trolamine salicylate), titanium dioxide, and zinc oxide. One skilled in the art will appreciate that other sunscreen agents may be used in the compositions and preparations of the present invention.

The skin-lightening agents included in compositions used for certain embodiments include ascorbic acid and derivatives thereof, kojic acid and derivatives thereof, hydroquinone, azelaic acid, and various plant extracts such as those from licorice, grape seed, and bear berry. Those skilled in the art will appreciate that other skin-lightening agents may be included in the compositions used for some of the methods of the present invention.

As noted above, conditioning agents are also included in the compositions used for other particular embodiments directed to the methods of the present invention. These agents comprise substances that enhance the appearance of dry or damaged skin, as well as materials that adhere to the skin to reduce flaking, restore suppleness, and generally improve the appearance of skin. Representative examples of the skin conditioning agents that may be used include: acetyl cysteine, N-acetyl dihydrosphingosine, acrylates/behenyl acrylate/dimethicone acrylate copolymer, adenosine, adenosine cyclic phosphate, adensosine phosphate, adenosine triphosphate, alanine, albumen, algae extract, allantoin and deriviatives, aloe barbadensis extracts, aluminum PCA, amyloglucosidase, arbutin, arginine, azulene, bromelain, buttermilk powder, butylene glycol, caffeine, calcium gluconate, capsaicin, carbocysteine, carnosine, beta-carotene, casein, catalase, cephalins, ceramides, chamomilla recutita (matricaria) flower extract, cholecalciferol, cholesteryl esters, coco-betaine, coenzyme A, corn starch modified, crystallins, cycloethoxymethicone, cysteine DNA, cytochrome C, darutoside, dextran sulfate, dimethicone copolyols, dimethylsilanol hyaluronate, DNA, elastin, elastin amino acids, epidermal growth factor, ergocalciferol, ergosterol, ethylhexyl PCA, fibronectin, folic acid, gelatin, gliadin, beta-glucan, glucose, glycine, glycogen, glycolipids, glycoproteins, glycosaminoglycans, glycosphingolipids, horseradish peroxidase, hydrogenated proteins, hydrolyzed proteins, jojoba oil, keratin, keratin amino acids, and kinetin.

Other examples of skin conditioning agents that may be included in the compositions used for the present invention are: lactoferrin, lanosterol, lauryl PCA, lecithin, linoleic acid, linolenic acid, lipase, lysine, lysozyme, malt extract, maltodextrin, melanin, methionine, mineral salts, niacin, niacinamide, oat amino acids, oryzanol, palmitoyl hydrolyzed proteins, pancreatin, papain, PEG, pepsin, phospholipids, phytosterols, placental enzymes, placental lipids, pyridoxal 5-phosphate, quercetin, resorcinol acetate, riboflavin, RNA, saccharomyces lysate extract, silk amino acids, sphingolipids, stearamidopropyl betaine, stearyl palmitate, tocopherol, tocopheryl acetate, tocopheryl linoleate, ubiquinone, vitis vinifera (grape) seed oil, wheat amino acids, xanthan gum, and zinc gluconate. Skin conditioning agents, other than those listed above, may also be used, as is readily appreciated by those skilled in the art.

Other embodiments employ compositions that include at least one skin protectant, defined herein as a compound that protects injured or exposed skin or mucous membrane surfaces from harmful or irritating external compounds. Representative examples include: algae extract, allantoin, aluminum hydroxide, aluminum sulfate, betaine, camellia sinensis leaf extract, cerebrosides, dimethicone, glucuronolactone, glycerin, kaolin, lanolin, malt extract, mineral oil, petrolatum, potassium gluconate, and talc. Those skilled in the art will readily appreciate that skin protectants, other than those listed above, may be included in the compositions used for the methods of the present invention.

Yet further embodiments use compositions that comprise one or more emollients. An emollient, as the term is used herein, is a cosmetic ingredient that can help skin maintain a soft, smooth, and pliable appearance. Emollients are able to provide these benefits, largely owing to their ability to remain on the skin surface or in the stratum corneum to act as a lubricant and reduce flaking. Some examples of emollients, suitable for embodiments of this invention, are: acetyl arginine, acetylated lanolin, algae extract, apricot kernel oil PEG-6 esters, avocado oil PEG-11 esters, bis-PEG-4 dimethicone, butoxyethyl stearate, C₁₈-C₃₆ acid glycol ester, C₁₂-C₁₃ alkyl lactate, caprylyl glycol, cetyl esters, cetyl laurate, coconut oil PEG-10 esters, di-C₁₂-C₁₃ alkyl tartrate, diethyl sebacate, dihydrocholesteryl butyrate, dimethiconol, dimyristyl tartrate, disteareth-5 lauroyl glutamate, ethyl avocadate, ethylhexyl myristate, glyceryl isostearates, glyceryl oleate, hexyldecyl stearate, hexyl isostearate, hydrogenated palm glycerides, hydrogenated soy glycerides, hydrogenated tallow glycerides, hydroxypropyl bisisostearamide MEA, isostearyl neopentanoate, isostearyl palmitate, isotridecyl isononanoate, laureth-2 acetate, lauryl polyglyceryl-6 cetearyl glycol ether, methyl gluceth-20 benzoate, mineral oil, myreth-3 palmitate, octyldecanol, octyidodecanol, odontella aurita oil, 2-oleamido-1,3 octadecanediol, palm glycerides, PEG avocado glycerides, PEG castor oil, PEG-22/dodecyl glycol copolymer, PEG shorea butter glycerides, phytol, raffinose, stearyl citrate, sunflower seed oil glycerides, and tocopheryl glucoside. Those skilled in the art will readily appreciate that emollients, other than those listed above, may also be used.

Humectants may also be included in the compositions used for the methods of additional particular embodiments. Humectants are cosmetic ingredients that help maintain moisture levels in skin. Some examples of suitable humectants are: acetyl arginine, algae extract, aloe barbadensis leaf extract, betaine, 2,3-butanediol, chitosan lauroyl glycinate, diglycereth-7 malate, diglycerin, diglycol guanidine succinate, erythritol, fructose, glucose, glycerin, honey, hydrolyzed wheat protein/PEG-20 acetate copolymer, hydroxypropyltrimonium hyaluronate, inositol, lactitol, maltitol, maltose, mannitol, mannose, methoxy PEG, myristamidobutyl guanidine acetate, polyglyceryl sorbitol, potassium PCA, propylene glycol, sodium PCA, sorbitol, sucrose, and urea. Other humectants may be used for yet additional embodiments of this invention, as will be appreciated by those skilled in the art.

In addition to the active agents disclosed above, the compositions employed in the methods of certain embodiments of the present invention may also contain inert, physiologically acceptable carriers or diluents. Suitable carriers or diluents include, but are not limited to: water, physiological saline, bacteriostatic saline (e.g., saline containing 0.9 mg/ml benzyl alcohol), petrolatum based creams (e.g., USP hydrophilic ointments and similar creams), various types of pharmaceutically acceptable gels, and short chain alcohols and glycols (e.g., ethyl alcohol and propylene glycol).

Disclosed methods may, in some embodiments, employ compositions that comprise additional ingredients such as fatty alcohols, fatty acids, organic or inorganic bases, preserving agents, wax esters, steroid alcohols, triglyceride esters, phospholipids such as lecithin and cephalin, polyhydric alcohol esters, fatty alcohol ethers, hydrophilic lanolin derivatives, hydrophilic beeswax derivatives, cocoa butter waxes, silicon oils, pH balancers, cellulose derivatives, and hydrocarbon oils such as palm oil, coconut oil, and mineral oil. Other additional ingredients that are particularly useful, as is well understood by those skilled in the art, are those that may be used to vary the texture, viscosity, color and appearance of the above compositions and preparations, and include emulsifying agents, thickening agents, and surfactants.

More specifically, emulsifiers and surfactants may be included in those compositions used for the present invention that are formulated as emulsions. Either water in oil or oil in water emulsions may be formulated. Examples of suitable surfactants and emulsifying agents include: nonionic ethoxylated and nonethoxylated surfactants, abietic acid, almond oil PEG, beeswax, butylglucoside caprate, C₁₈-C₃₆ acid glycol ester, C₉-C₁₅ alkyl phosphate, caprylic/capric triglyceride PEG-4 esters, ceteareth-7, cetyl alcohol, cetyl phosphate, corn oil PEG esters, DEA-cetyl phosphate, dextrin laurate, dilaureth-7 citrate, dimyristyl phosphate, glycereth-17 cocoate, glyceryl erucate, glyceryl laurate, hydrogenated castor oil PEG esters, isosteareth-11 carboxylic acid, lecithin, lysolecithin, nonoxynol-9, octyldodeceth-20, palm glyceride, PEG diisostearate, PEG stearamine, poloxamines, polyglyceryls, potassium linoleate, PPG's, raffinose myristate, sodium caproyl lactylate, sodium caprylate, sodium cocoate, sodium isostearate, sodium tocopheryl phosphate, steareths, TEA-C₁₂-C₁₃ pareth-3 sulfate, tri-C₁₂-C₁₅ pareth-6 phosphate, and trideceths. Other surfactants and emulsifiers may be used, as will be appreciated by those skilled in the art.

In further embodiments of this invention, directed to methods for treating hyperpigmentation of the skin, the compositions used therefor also comprise thickening or viscosity increasing agents. Suitable examples include those agents commonly used in skin care preparations, such as: acrylamides copolymer, agarose, amylopectin, bentonite, calcium alginate, calcium carboxymethyl cellulose, carbomer, carboxymethyl chitin, cellulose gum, dextrin, gelatin, hydrogenated tallow, hydroxytheylcellulose, hydroxypropylcellulose, hydroxpropyl starch, magnesium alginate, methylcellulose, microcrystalline cellulose, pectin, various PEG's, polyacrylic acid, polymethacrylic acid, polyvinyl alcohol, various PPG's, sodium acrylates copolymer, sodium carrageenan, xanthan gum, and yeast beta-glucan. Thickening agents other than those listed above may also be used in related embodiments of the present invention.

As heretofore noted, the compositions used for the methods of the present invention, being products for topical application to human skin, are, accordingly, formulated as a cream, gel, fluid cream or milk, lotion, or oil. Also, the above compositions may be further combined with suitable excipients adapted for application to the face and neck. Suitable excipients should have a high affinity for the skin, be well tolerated, stable, and yield a consistency that allows for easy and pleasant utilization.

As a more specific example of a method of the present invention, aside from the content of the composition used, a small amount of the composition (from about 1 ml to about 5 ml) is applied to exposed areas of skin from a suitable container or applicator, and, if necessary, the composition is then spread over and/or rubbed into the skin using the hand, finger, or other suitable device. Each composition disclosed herein is typically packaged in a container that is appropriate in view of its viscosity and intended use by the consumer. For example, a lotion or fluid cream may be packaged in a bottle, roll-ball applicator, capsule, propellant-driven aerosol device, or a container fitted with a manually operated pump. A cream may simply be stored in a non-deformable bottle, or in a squeeze container, such as a tube or a lidded jar.

The following examples are provided for the purpose of illustration, not limitation.

### EXAMPLES

The following examples illustrate the preparation, characterization and utility of certain compositions used for exemplary embodiments directed to the methods of the present invention.

### Example 1

### Representative Moisturizing Lotion

| Ingredients | % w/w |
|---|---|
| water | 73.80% |
| glycerin | 1.00% |
| xanthan gum | 0.50% |
| diisopropyl adipate | 4.00% |
| isocetyl stearate | 6.00% |
| octyl palmitate | 10.00% |
| glyceryl stearate | 1.00% |
| cetyl alcohol | 1.00% |
| stearyl alcohol | 0.80% |
| behenyl alcohol | 0.50% |
| palmitic acid | 0.25% |
| stearic acid | 0.25% |
| glycyl-L-histidyl-L-lysine copper complex | 0.30% |
| propylene glycol | 0.55% |
| diazolidinyl urea | 0.03% |
| iodopropynyl butylcarbonate | 0.02% |
| total | 100.00% |

### Example 2

### Representative Moisturizing Cream

| Ingredients | % w/w |
|---|---|
| purified water | 77.35% |
| ethylhexyl palmitate | 8.00% |
| octyldodecanol | 2.50% |
| butyloctyl calicylate | 2.00% |
| squalane | 1.50% |
| jojoba oil | 0.50% |
| tocopheryl acetate | 0.20% |
| bisabolol | 0.20% |
| polyacrylamide | 1.50% |
| laureth-7 | 0.50% |
| glycerin | 3.00% |
| panthenol | 0.60% |
| allantion | 0.10% |
| cyclomethicone | 0.50% |
| carbomer | 0.10% |
| polysorbate 20 | 0.20% |
| L-alanyl-L-histidyl-L-lysine copper | 0.25% |
| complex | |
| propylene glycol | 0.56% |
| diazolidinyl urea | 0.30% |
| methylparaben | 0.11% |
| propylparaben | 0.03% |
| total | 100.00% |

### Example 3

### Representative Body Lotion

| Ingredients | % w/w |
|---|---|
| water | 74.40% |
| hydrogenated vegetable oil | 6.00% |
| canola oil | 4.00% |
| polyoxyethylene stearyl stearate | 4.00% |
| steareth-21 | 2.00% |
| octyldodecanol | 6.00% |
| sorbeth-30 | 2.50% |
| glycyl-L-histidyl-L-lysine copper complex | 0.10% |
| propylene glycol | 0.56% |
| diazolidinyl urea | 0.30% |
| methylparaben | 0.11% |
| propylparaben | 0.03% |
| Total | 100.00% |

### Example 4

### Representative Water-In-Oil Emulsion

| Ingredients+ | % w/w |
|---|---|
| purified water | 71.84% |
| quarternium 82 | 2.00% |
| polyquarternium-37 | 1.10% |
| mineral oil | 0.50% |
| PPG-1-trideceth-6 | 0.40% |
| ethylhexyl isononanoate | 20.00% |
| cetyl dimethicone copolyol | 1.00% |
| L-alanyl-L-histidyl-L-lysine copper | 0.10% |
| complex | |
| Kojic Acid | 2.0% |
| propylene glycol | 0.56% |
| imidazolidinyl urea | 0.30% |
| methylparaben | 0.11% |
| propylparaben | 0.03% |
| butylparaben | 0.02% |
| isopropylparaben | 0.02% |
| isobutylparaben | 0.02% |
| Total | 100.00% |

### Example 5

### Representative Oil-In-Water Emulsion Type Face Cream

| Ingredients | % w/w |
|---|---|
| purified water | 75.20% |
| glycerin | 4.00% |
| steareth-100 | 0.60% |
| steareth-2 | 0.35% |
| xanthan gum | 0.35% |
| isopropyl palmitate | 4.00% |
| isohexanodecane | 1.00% |
| isostearyl isostearate | 1.20% |
| octyl dodecanol | 1.00% |
| stearic acid | 2.50% |
| cetostearyl alcohol | 2.50% |
| petrolatum | 4.00% |
| glycyl-L-histidyl-L-lysine copper | 0.10% |
| complex | |
| phenoxyethanol | 3.00% |
| methylparaben | 0.11% |
| propylparaben | 0.03% |
| butylparaben | 0.02% |
| isopropylparaben | 0.02% |
| isobutylparaben | 0.02% |
| Total | 100.00% |

### Example 6

### Representative High Silicon Content Cream

| Ingredients | % w/w |
|---|---|
| purified water | 44.25% |
| dimethicone | 50.00% |
| behentriomnium methosulfate | 4.00% |
| cetearyl alcohol | 2.00% |
| glycyl-L-histidyl-L-lysine copper | 0.20% |
| complex | |
| methylparaben | 0.30% |
| ethylparaben | 0.10% |
| propylparaben | 0.03% |
| butylparaben | 0.02% |
| Total | 100.00% |

### Example 7

The utility of an example of the disclosed compositions of the present invention was demonstrated in an 8 week study involving twelve female human subjects having ages ranging from 41 to 55 years and a mean age of 46. Each of the subjects applied a moisturizing cream similar to that of Example 2, except for containing 0.25% glycyl-L-histidyl-L-lysine copper complex instead of L-alanyl-L-histidyl-L-lysine copper complex. The cream was applied twice each day—once in the morning and once at night. At the beginning of the study, and at week 8, the amount and extent of hyperpigmentation was assessed by a clinician using the following scale.

| Rating | Category |
|---|---|
| 0 | None |
| 1-3 | Mild |
| 4-6 | Moderate |
| 7-9 | Severe |

At the beginning of the study, the subjects had a mean score of 3.17 for mottled hyperpigmentation. At the end of the study the mean scoring for hyperpigmentation was 1.92, a significant 40% reduction from the baseline assessment. The subjects were also asked to perform a self-assessment of the change in hyperpigmentation at weeks 4 and 8. More than 50% of the subjects agreed that the appearance of blotchy discolorations, as well as age/brown spots, was reduced relative to the baseline as early as 4 weeks after the treatment began.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing, it will be appreciated that, although specific embodiments of the present invention have been described herein for purposes of illustration, various modifications may be made without deviating from the spirit and scope of the invention. Accordingly, the present invention is not limited except as by the appended claims.

1. A method for treating hyperpigmentation of skin comprising contacting skin in need thereof with an effective amount of a composition wherein the composition comprises a peptide copper complex.

2. The method of claim 1 wherein the peptide copper complex is L-alanyl-L-histidyl-L-lysine:copper(II).

3. The method of claim 1 wherein the peptide copper complex is L-valyl-L-histidyl-L-lysine:copper(II).

4. The method of claim 1 wherein the peptide copper complex is glycyl-L-histidyl-L-lysine:copper(II).

5. The method of claim 1 wherein the molar ratio of peptide to copper in the peptide copper complex ranges from about 1:1 to about 3:1.

6. The method of claim 1 wherein the molar ratio of peptide to copper in the peptide copper complex ranges from about 1:1 to about 2:1.

7. The method of claim 1 wherein the peptide copper complex is present at a concentration ranging from about 0.01% to about 5% by weight of the composition.

8. The method of claim 1 wherein the peptide copper complex is present at a concentration ranging from about 0.025% to about 1% by weight of the composition.

9. The method of claim 1 wherein the concentration of the peptide copper complex is present at a concentration ranging from about 0.05% to about 0.5% by weight of the composition.

10. The method of claim 1 wherein the peptide copper complex is encapsulated in a liposome or microsponge adapted to aid in the delivery of the peptide copper complex to the area of the skin or to enhance the stability of the skin care composition.

11. The method of claim 1 wherein the peptide copper complex is formulated in an instrument adapted to deliver the peptide copper complex to the area of the skin via iontophoresis.

12. The method of claim 1 wherein the composition further comprises an inert and physiologically-acceptable carrier or diluent.

13. The method of claim 12 wherein the inert and physiologically-acceptable carrier or diluent is water, physiological saline, bacteriostatic saline, a petrolatum based cream, a pharmaceutically acceptable gel, a short chain alcohol, or a short chain glycol.

14. The method of claim 1 wherein the composition further comprises a skin-lightening agent.

15. The method of claim 1 wherein the composition further comprises a sunscreen agent.

16. The method of claim 1 wherein the composition further comprises a skin conditioning agent.

17. The method of claim 1 wherein the composition further comprises a skin protectant.

18. The method of claim 1 wherein the composition further comprises an emollient.

19. The method of claim 1 wherein the composition further comprises a humectant.

20. The method of claim 1 wherein the composition further comprises a fatty alcohol, a fatty acid, an organic base, an inorganic base, a preserving agent, a wax ester, a steroid alcohol, a triglyceride ester, a phospholipid, a polyhydric alcohol ester, a fatty alcohol ether, a hydrophilic lanolin derivative, a hydrophilic beeswax derivative, a cocoa butter wax, a silicon oil, a pH balancer, a cellulose derivative, a hydrocarbon oil, or a mixture thereof.

21. The method of claim 1 wherein the composition further comprises an emulsifying agent, a surfactant, a thickening agent, an excipient, or a mixture thereof.

22. The method of claim 1 wherein the composition is in the form of a solution, cream, gel, fluid cream, lotion, or oil.

23. The method of claim 1 wherein the composition further comprises retinol, a retinol derivative, or a mixture thereof.
